(19) United States
(12) Patent Application Publication
(10) Pub. No.: US 2016/0207894 A1
(43) Pub. Date: Jul. 21, 2016

(54) Synthesis of Diacids, Dialdehydes, or Diamines from THF-Diols

(71) Applicant: ARCHER DANIELS MIDLAND COMPANY, Decatur, IL (US)

(72) Inventor: Kenneth STENSRUD, Decatur, IL (US)

(21) Appl. No.: 14/914,284

(22) PCT Filed: Sep. 25, 2014
(86) PCT No.: PCT/US2014/057399

§ 371 Date: Feb. 25, 2016

Related U.S. Application Data
(60) Provisional application No. 61/891,928, filed on Oct. 17, 2013.

(51) Int. Cl. C07D 307/16; C07D 307/14; C07D 307/12

(52) U.S. Cl. CPC C07D 307/16; C07D 307/12; C07D 307/14

(57) ABSTRACT

A simple and elegant chemical process for the synthesis of oxygenated products, such as acids and aldehydes, or other derivative products, such as amines and nitriles, of cyclic bifunctional molecules made from renewable, bio-based sources such as HMF and/or its reduction product, 2,5-bis(hydroxymethyl)-tetrahydrofuran (bHMTHF) is described. In general, the process involves: a) generating a tetrahydrofuran-2,5-diyl-bis(methylene)-bis(sulfonate) from bHMTHFs using a sulfonate; b) displacing nucleophilically at least a sulfonate leaving group from the tetrahydrofuran-2,5-diyl-bis(methylene)-bis(sulfonate) to form a THF-dinitrile; and either c) oxidizing the THF-dinitrile with an acid having a pKa of ≦0 to generate a di-acid, or d) reducing partially the THF-dinitrile to generate a di-aldehyde, or e) reducing fully the THF-dinitrile to generate a di-amine.

### PRIORITY CLAIM

This Application claims benefit of priority from U.S. Provisional Application No. 61/891,928, filed on Oct. 17, 2013.

### FIELD OF INVENTION

The present application is in the field of art relating to cyclic bifunctional materials useful as monomers in polymer synthesis and as intermediates generally, and to the methods by which such materials are made. In particular, the present invention pertains to synthesis of nitriles, carboxylic acids, aldehydes, and amines from renewable biomass resources.

### BACKGROUND

In recent years, interest has grown in renewable source-based alternatives for cyclic bi-functional materials that have been prepared conventionally from petroleum or fossil-based hydrocarbons, as petroleum resources become increasingly scarce and costly. As an abundant bio-based or renewable-resource, carbohydrates represent a viable alternative feedstock for producing such materials. Biomass contains carbohydrates or sugars (i.e., hexoses and pentoses) that can be converted into value added products from renewable hydrocarbon sources.

In recent years, researchers have directed their efforts towards discovering efficacious processes for converting biomass into sustainable feedstock for various versatile organic chemical platforms. When considering possible downstream chemical processing technologies, the conversion of sugars to value-added chemicals is very important. Recently, the production of furan derivatives from sugars has become exciting in chemistry because of the potential for achieving sustainable energy supply and chemicals production.

As an important intermediate substance, readily made from carbohydrates, the compound 5-(hydroxymethyl)-furan-2-carbaldehyde (HMF) exemplifies a multifaceted substrate. HMF is a suitable starting material for the formation of various furan ring derivatives that are intermediates for chemical syntheses, and as potential substitutes for benzene-based rings compounds that have been derived ordinarily from petroleum resources. Recent developments in large-scale manufacturing technology have permitted HMF to become more available commercially. This advance affords opportunities for various secondary or derivative products to be made, which can increase the potential for value added chemical compounds without incurring inordinate costs. HMF, however, has limited uses as a chemical per se, other than as a source for making derivatives. Moreover, HMF itself is rather unstable and tends to polymerize and or oxidize with prolonged storage. Due to the instability and limited applications of HMF itself, studies have broadened to include the synthesis and purification of a variety of HMF derivatives.

Catalyzed complete reduction (hydrogenation) of HMF A, such as depicted in Scheme 1, under mild conditions generates THF-diols, also known by their IUPAC names: ((2R,5S)-tetrahydrofuran-2,5-diyl)dimethanol B and ((2S,55)-tetrahydrofuran-2,5-diyl)dimethanol C (collectively regarded as 2,5-bishydroxymethyl-tetrahydrofurans (also referred to herein as bHMTHFs)), in a 90:10 cis:trans diastereomeric mixture.

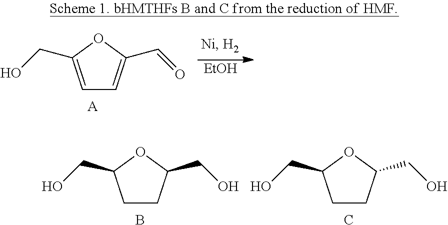

bHMTHFs are versatile molecules that when modified can serve as a substitute for a variety of structurally analogous molecules that have conventionally been derived from petroleum-based sources.

Heretofore, research for chemical derivatives using bHMTHFs has received limited attention due in part to the great cost and relative paucity (e.g., ˜$200 per gram commercially) of the compounds. Recently, a need has arisen for a way to unlock the potential of bHMTHFs and their derivative compounds, as these chemical entities have gained attention as valuable glycolic antecedents for the preparation of polymers, solvents, additives, lubricants, and plasticizers, etc. Furthermore, the inherent, immutable chirality of bHMTHFs makes these compounds useful as potential species for pharmaceutical applications or candidates in the emerging chiral auxiliary field of asymmetric organic synthesis. Given the potential uses, a cost efficient and simple process that can synthesis derivatives from bHMTHFs would be appreciated by manufacturers of both industrial and specialty chemicals alike as a way to better utilize biomass-derived carbon resources.

### SUMMARY OF THE INVENTION

The present disclosure describes, in part, a simple and elegant chemical process for the synthesis of oxygenated products, such as acids and aldehydes, or other derivative products, such as amines and nitriles, of cyclic bifunctional molecules made from renewable, bio-based sources such as HMF and/or its reduction product, 2,5-bis(hydroxymethyl) tetrahydrofuran (bHMTHF). In general, the process encompasses: a) derivatizing bHMTHF using a sulfonate to generate a tetrahydrofuran-2,5-diyl-bis(methylene)-bis(sulfonate); b) displacing at least a sulfonate leaving group from the tetrahydrofuran-2,5-diyl-bis(methylene)-bis(sulfonate) with a nucleophile; and either c) hydrolyzing fully with a strong Brønsted acid having a pKa of ≦0 to generate a di-acid, or d) reducing partially to generate a di-aldehyde, or e) reducing fully to generate a di-amine

In another aspect, the present inventive concept also encompasses the different cis and trans isomeric precursors or intermediates, and products of the present process:

When cyanide is used in the nucleophilic displacement of the sulfonate leaving group, one generates THF-2,5-diacetonitriles: 2,2′-((2R,5S)-tetrahydrofuran-2,5-diyl)diacetonitrile A and 2,2′-((2S,5S)-tetrahydrofuran-2,5-diyl)diacetonitrile B:

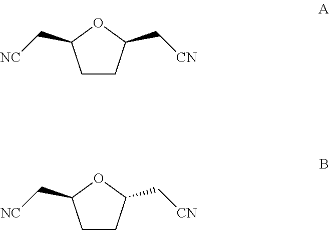 [SMILES: N#CC[C@@H]1CC[C@H](CC#N)O1 ; N#CC[C@@H]1CC[C@@H](CC#N)O1]

When one oxidizes THF-2,5-diacetonitriles with an acid, one generates THF-2,5-diacetic acids: 2,2′-((2R,5S)-tetrahydrofuran-2,5-diyl)diacetic acid A, and 2,2′-((2S,5S)-tetrahydrofuran-2,5-diyl)diacetic acid B:

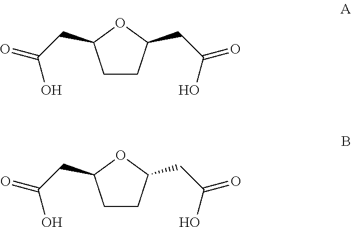 [SMILES: O=C(O)C[C@H]1CC[C@@H](CC(=O)O)O1 ; O=C(O)C[C@@H]1CC[C@@H](CC(=O)O)O1]

When one reduces selectively THF-2,5-diacetonitriles, one generates THF-2,5-diacetaldehydes: 2,2′-((2R,5S)-tetrahydrofuran-2,5-diyl)diacetaldehyde A, and 2,2′-((2S,5S)-tetrahydrofuran-2,5-diyl)diacetaldehyde B:

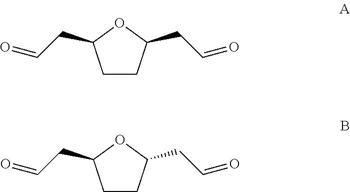 [SMILES: O=CC[C@@H]1CC[C@H](CC=O)O1 ; O=CC[C@@H]1CC[C@@H](CC=O)O1]

When one reduces completely THF-2,5-diacetonitriles, one generates THF-2,5-diamines: 2,2′-((2R,5S)-tetrahydrofuran-2,5-diyl)diethanamine A, and 2,2′-((2S,5S)-tetrahydrofuran-2,5-diyl)diethanamine B:

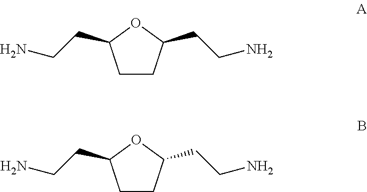 [SMILES: NCC[C@@H]1CC[C@H](CCN)O1 ; NCC[C@@H]1CC[C@@H](CCN)O1]

Additional features and advantages of the present purification process will be disclosed in the following detailed description. It is understood that both the foregoing summary and the following detailed description and examples are merely representative of the invention, and are intended to provide an overview for understanding the invention as claimed.

### DETAILED DESCRIPTION OF THE INVENTION

### Section I.—Description

The present synthesis process opens a new pathway for potential industrial, large-volume production of either 1) an oxidation product, 2) a partially reduced product, or 3) a fully reduced product from THF-diols. In general, the overall preparation process for the different products involves a three-step reaction sequence. Common to each of the product synthesis, the first two reactions steps generate diacetonitrile variants from THF-diols. The third reaction step can vary depending on the desired products; in particular, when the diacetonitrile species is oxidized, one generates a corresponding di-acetic acid species; when the diacetonitrile is partially or selectively reduced, one produces a corresponding di-aldehyde species; and when the diacetonitrile species is fully or completely reduced, one makes a corresponding diethyl-amine species.

For instance, according to an embodiment for oxygenated products, as illustrated in Scheme 2, THF-diol is derivatized first by sulfonation; second, the resultant disulfonate is reacted with a nucleophile which displaces a sulfonate leaving group; and third, the resultant di-nitrile is either oxidized or reduced partially to generate, respectively, either a di-acid or di-aldehyde. The process is performed under relatively mild conditions (e.g., about −20° C. or −10° C. to about 150° C., depending on reagents) and produces good yields of better than 50% or 60% conversion of THF-diols into the corresponding acids or aldehydes.

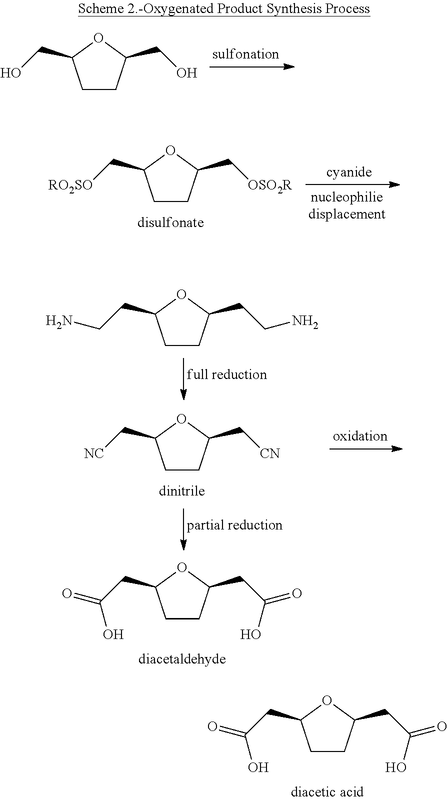

In another embodiment also shown in Scheme 2, following the first two reaction steps, one makes a di-amine, when the di-nitrile species is completely reduced. Scheme 3 illustrates this full reduction step. The process produces a significant yield of more than 50% of the di-amine species.

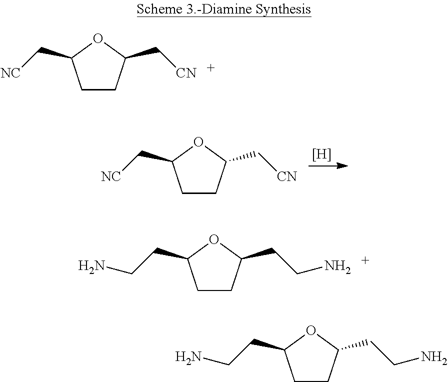

The THF-diacetonitrile species is a versatile precursor to corresponding THF-diacetic acids, THF-diacetaldehydes, or THF-diamines In particular, the resulting compounds can be, for example: a) 2,2′-((2R,5S)-tetrahydrofuran-2,5-diyl)diacetic acid and 2,2′-((2S,5S)-tetrahydrofuran-2,5-diyl)diacetic acid, (collectively, THF-2,5-diacetic acids); b) 2,2′4(2R,5S)-tetrahydrofuran-2,5-diyl)diacetaldehyde and 2,2′-((2S,5S)-tetrahydrofuran-2,5-diyl)diacetaldehyde (collectively, THF-2,5-diacetaldehydes); or c) 2,2′-((2R,5S)-tetrahydrofuran-2,5-diyl)diethanamine and 2,2′-((2S,5S)-tetrahydrofuran-2,5-diyl)diethanamine (collectively, THF-2,5-diamines).

It is envisioned that each of these resulting compounds can serve as a chemical platform or feedstock; that is, useful building blocks in various applications, such as polymer synthesis or as a precursor for various other chemical and industrial materials. The fixed chiral centers of these precursors are also attractive features. As these analogs gain more traction as value-added chemicals, novel derivatives thereof, tertiary products will likely be studied further to divulge sui generis properties with industrial potential.

### A. Sulfonation

To generate the disulfonates of bHMTHF in the sulfonation reaction of the present preparation process, one can use a variety of sulfonates, including but not limited to, mesylate (methanesulfonate), CH₃SO₂O—

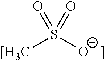

(—OMs); triflate (trifluoromethanesulfonate), CF₃SO₂O—

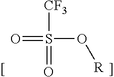

(—OTfs); tosylate (p-toluenesulfonate), CH₃C₆H₄SO₂O—

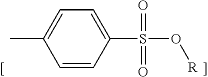

(—OTs); esylate (ethanesulfonate), C₂H₅SO₂O—

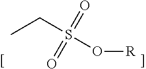

(—OEs); besylate (benzenesulfonate), C₆H₅SO₂O—

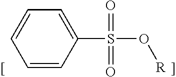

(—OBs), and other alkyl and aryl sulfonates without limitation.

As the most powerful leaving group, triflates (TfO) are more preferred. This reaction exhibits relatively fast kinetics and generates an activated triflic complex. The reaction is usually conducted at a low temperature, less than 0° C. (e.g., typically about −10° C. or −12° C. to about −20° C. or −25° C.), to control the reaction kinetics more easily. This reaction is essentially irreversible, as the liberated triflate is entirely non-nucleophilic. The triflic complex then reacts readily with the bHMTHF, forming an bHMTHF-triflate with concomitant release and protonation of a nucleophilic base (e.g., pyrimidine, dimethyl-aminopyridine, imidazole, pyrrolidine, and morpholine).

The tosylate, mesylate, brosylate, benzenesulfonate, ethylsulfonate or other sulfonate species can be as effective as triflate in imparting nucleofuges, and manifesting overall yields that were commensurate with that achieved with triflate. But, these other sulfonates tend to react more slowly in comparison to the triflate. To compensate for this, operations at higher temperatures are typically needed for better yields when using these other species.

In the reaction of bHMTHFs with sulfonates to corresponding disulfonates, the operative temperature parameters for these other sulfonate species can be from about 0° C. to about 50° C., over a reaction time of at least 5-6 hours, in some examples up to about 24 hours. In some embodiments, the reaction step can be performed at or near ambient room temperature (e.g., about 10° C., 15° C. or 20° C. to about 30° C. or 40° C.; typically about 17° C. or 18° C. to about 22° C., 25° C. or 27° C.), depending on the particular species.

Overall, the present synthesis process can produce copacetic yields of disulfonates of bHMTHF, as demonstrated in the accompanying examples. The process enables the production of disulfonates of bHMTHF in reasonably high molar yields of at least 50% from the bHMTHF, typically more than 55% or 60%. With proper control of the reaction conditions and time, disulfonates of bHMTHF are produced at yields of ≧70%, typically ≧80% or 90% or better. The THF-diol or HMF starting materials can be obtained either commercially or synthesized from relatively inexpensive, widely-available biologically-derived feedstocks. (For analogous reaction, see, U.S. Provisional Application No. 61/816,847, K. Stensrud, “5-(Hydroxymethyl) Furan-2-Carbaldehyde (HMF) Sulfonates and Process for Synthesis Thereof,” filed Apr. 29, 2013, the content of which is incorporated herein by reference.)

### B. Nucleophilic Displacement

Nucleophilic displacement occurs in at least two parts of the synthesis process. First, as mentioned above during the sulfonation reaction, the bHMTHF release and protonates a nucleophilic base. In an embodiment, the nucleophile is a nitrogen-centered compound, such as pyrimidine, which is used as a base to catalyze the conversion of bHMTHF to its corresponding disulfonate.

Second, the disulfonates of bHMTHF is reacted with another nucleophile, which according to an embodiment, is a cyanide. (After the diacetonitrile is generated with a cyanide nucleophile, the bHMTHFs themselves can also be referred to as cyanide-derivatized bHMTHFs.) The cyanide species can be a cyanide salt, for example including but not limited to, lithium cyanide, sodium cyanide, potassium cyanide, trimethylsilyl cyanide, cesium cyanide, tetrabutyl ammonium cyanide, tetraethylammonium cyanide, copper (I) cyanide, silver cyanide, gold cyanide, mercury (II) cyanide, zinc cyanide, platinum (II) cyanide, palladium (II) cyanide, cobalt (II) cyanide. Although each of these cyanide species are effective in forming the THF-2,5-diacetonitriles precursor from THF-2,5-disulfonates in high yields (e.g., >85% or 90%), more commonly one would employ the potassium or sodium cyanide, trimethylsilyl cyanide, tetrabutyl ammonium cyanide, silver cyanide, and copper cyanide species, because of cost and availability. In certain examples, KCN is a more favored species, as potassium exhibits greater reactivity as a stronger anion than sodium.

When reacted with the cyanide the THF-disulfonates convert to a 9:1 diastereomeric mixture of THF-2,5-diacetonitriles. The yield of THF-2,5-diacetonitriles is greater than 70% or 75%, typically ≧80% or 90% or more.

In the conversion of THF-2,5-disulfonates to corresponding diacetonitriles, the solvent used has a boiling point of at least 75° C. up to about 200° C. This is desired because, as in certain embodiments, the reaction temperatures may span from about 120° C. to about 175° C., typically from about 110° C. to about 150° C., although other temperatures either higher or lower (e.g., about 80° C., 95° C. or 100° C. to about 140° C. or 190° C.; typically about 90° C. or 110° C. to about 130° C. or 150° C., 170° C. or 180° C.) are also possible.

### C. Solvent & Operational Conditions

In the present synthesis process, aprotic solvents are favored, as they let the nucleophile be exposed, with little solvation, and hence enhances Sn2 reactions. In aprotic solvents a greater dielectric constant can help prevent the solvent from reacting with the primary reagents, hence minimizing formation of side-products.

The reactions of the present synthesis process are conducted in solvents with a relative permittivity ≧εᵣ 25, typically about 30 or 35. For example, DMSO and DMF exhibit relatively high dielectric constants (e.g., ˜30 or 32). Other solvents with high boiling points and dielectric constants, such as NMP and DMA, are effective in cyanide for sulfonate displacement reactions. The reaction to derivatize of bHMTHF with a sulfonate is performed in a solution of solvent having a boiling point ≧110° C.

The reactions herein are used in effectuating the quantitative conversion of bHMTHFs to corresponding disulfonates. In certain preferred embodiments, a solvent with dielectric constants of at least 30 or 35 are employed in the conversion of THF-2,5-disulfonates to corresponding THF-2,5-diacetonitriles.

### D. Products

In the third step of the present synthesis process, according to each respective embodiment when the THF-2,5-diacetonitriles is either oxidized or reduced, a 9:1 diastereomeric mixture of cis and trans bHMTHFs is converted to a 9:1 diastereomeric mixture of: THF-2,5-diacetic acids, THF-diacetaldehydes, or THF-diamines

The oxidative reaction produces corresponding THF-2,5-diacetic acids, such as illustrated in Scheme 4.

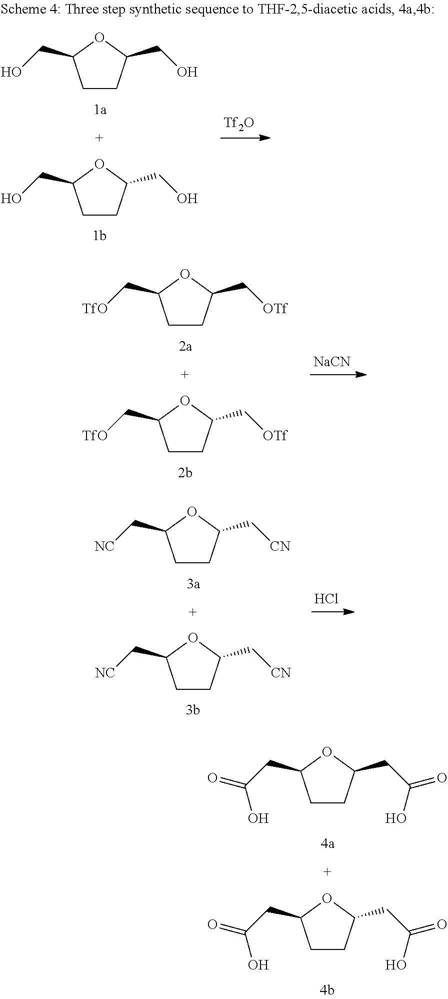

THF-2,5-diyl-diacetonitriles are subjected to hydrolysis with a concentrated aqueous Brønsted acid solution that effects the oxidized product. The strong Brønsted acid has a pKa of ≦0, which may include without limitation, for example: aqueous hydrochloric, hydrobromic, hydroiodic, perchloric, sulfuric, p-toluenesulfonic, triflic, methanesulfonic, or benzenesulfonic acids.

In the conversion of the THF-2,5-diacetonitriles to THF-2,5-diacetic acids, one can operate the reaction at a temperature from about 0° C. to about 100° C. The yield of THF-2,5-diacetic acids from THF-2,5-diacetonitriles is >85% or 90%.

A second class of product is formed when the THF-2,5-diacetonitriles are reduced partially to the corresponding THF-2,5-diacetaldehyde, through a transformation facilitated by the solvent medium. One can use reaction temperatures of about −78° C. to 110° C. The yield of THF-2,5-diacetaldehyde from THF-2,5-diacetonitrile is ≧50%. According to an embodiment, one can deploy a hindered organo-metalic (e.g., aluminum) hydride to selectively reduce THF-2,5-diacetonitriles to THF-2,5-diacetaldehydes; while in an alternative embodiment, a supported catalyst, such as nickel or palladium, is used. Concentrated formic acid is used as a solvent in the catalytic reduction of THF-2,5 diacetonitriles to THF-diacetaldehydes. The hydrogenation reaction usually involves operating at a hydrogen pressure that does not exceed about 250 psi in a reaction vessel. In an embodiment, an aqueous trifluoroacetic matrix is used as a solvent in the selective reduction of THF-2,5-diacetonitriles to THF-diacetaldehydes.

THF-diethyl-amines are the third class of compounds generated when the THF-2,5-diacetonitriles are reduced completely. The reaction is conducted with a reaction temperature range of about 0° C. to about 50° C. The yield of diethyl-amines from diacetonitriles can be ≧85% or 90%, usually 92% or greater. Pursuant to one embodiment, an unhindered, organometallic (e.g., lithium) hydride in an inert, water-free matrix is utilized for complete reduction of the THF-2,5-diacetonitriles to the corresponding THF-2,5-diethyl-amines; in another embodiment, a carbon supported palladium catalyst immured in an ethanolic matrix, saturated with hydrogen gas, is effective. The hydrogen pressure in these instances does not exceed about 1200 psi.

### Section II.—Examples

The present synthesis system is further illustrated in the following examples for making the A) di-acetic acid, B) di-acetaldehyde, and C) diethyl-amine products.

### A. Synthesis of THF-2,5-Diacetic Acid Isomers

Example 1, demonstrates one approach for synthesizing 2,2′-((2R,5S)-tetrahydrofuran-2,5-diyl)diacetic acid 4a and 2,2′-((2S,5S)-tetrahydrofuran-2,5-diyl)diacetic acid, 4b

Step 1: Synthesis of ((2R,5S)-tetrahydrofuran-2,5-diyl)bis(methylene) bis(trifluoromethanesulfonate) 2a, and ((2S,5S)-tetrahydrofuran-2,5-diyl)bis(methylene) bis(trifluoromethanesulfonate) 2b.

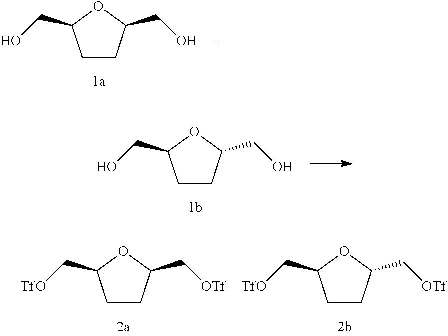 [SMILES: OC[C@@H]1CC[C@H](CO)O1 ; OC[C@@H]1CC[C@@H](CO)O1 ; O=S(=O)(OC[C@H]1CC[C@@H](COS(=O)(=O)C(F)(F)F)O1)C(F)(F)F ; O=S(=O)(OC[C@@H]1CC[C@@H](COS(=O)(=O)C(F)(F)F)O1)C(F)(F)F]

Experimental: An oven dried, 25 mL single-neck round bottomed flask equipped with a ½″×⅛″ tapered PTFE coated magnetic stir bar was charged with 226 mg of THF-diols 1 (1.71 mmol), 410 μL of pyridine (˜3 eq.) and 10 mL of anhydrous methylene chloride. The neck was capped with a rubber septum and a needle affixed to an argon inlet and the flask immersed in a saturated brine/ice bath (−10° C.). While stirring and under an argon blanket, 574 μL of triflic anhydride (3.42 mmol) was added dropwise over a 15 minute period. After complete addition, the flask was removed from the ice bath, warmed to ambient temperature, and the reaction continued for 2 more hours. After this time, an aliquot was removed and a portion spotted on a silica gel thin-layer chromatography plate abutting a spot from the THF diol starting materials for comparison. The plate was developed using a 100% ethyl acetate eluent, and after staining with cerium molydate, the product mixture revealed one distinct spot, R_{fl}=0.67 (THF-diol ditriflate). No band was observed at the baseline (R_{f}=0), indicating that all the THF-diol reagent had been converted. Solids were the filtered and the solvent removed under reduced pressure, affording 666 mg of 2a, 2b a yellow, viscous oil (98% of theoretical). ¹H NMR (CDCl₃, 400 MHz, salient cis isomer, 2a) δ (ppm) 4.58 (m 2H), 4.47 (m, 2H), 4.44 (m, 2), 4.32 (m, 2H), 2.15 (m, 2H), 1.87 (m, 2H); ¹³C NMR (CDCl₃, 100 MHz salient cis isomer) δ (ppm) 120.44, 84.2, 73.5, 30.3

Step 2: Synthesis of 2,2′-((2R,5S)-tetrahydrofuran-2,5-diyl)diacetonitrile 3a, and 2,2′-((2S,5S)-tetrahydrofuran-2,5-diyl)diacetonitrile 3b.

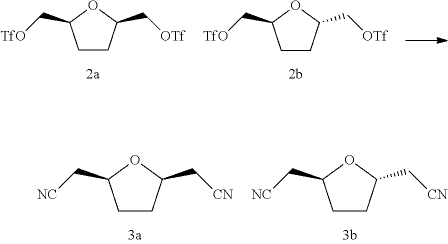 [SMILES: O=S(=O)(OC[C@H]1CC[C@@H](COS(=O)(=O)C(F)(F)F)O1)C(F)(F)F ; O=S(=O)(OC[C@@H]1CC[C@@H](COS(=O)(=O)C(F)(F)F)O1)C(F)(F)F ; N#CC[C@@H]1CC[C@H](CC#N)O1 ; N#CC[C@@H]1CC[C@@H](CC#N)O1]

Experimental: An oven dried An oven dried, 25 mL single-neck round bottomed flask equipped with a ½″×⅛″ tapered PTFE coated magnetic stir bar was charged with 650 mg of 2a and 2b (1.64 mmol), 161 mg of sodium cyanide (3.28 mmol), and 5 mL of anhydrous DMSO. The reaction was stirred vigorously overnight. After this time, an aliquot was removed and a portion spotted on a silica gel thin-layer chromatography plate abutting a spot from the 2a, 2b for comparison. The plate was developed using a 50% ethyl acetate in hexanes as the eluent, and after staining with cerium molybdate, the product mixture revealed one distinct spot, R_{fl}=0.58 (THF-diol ditriflate). No band was observed at R_{f}=0.41, corresponding to 2a, 2b, indicating that these reactants had been entirely converted. The solution was transferred to a 50 mL separatory funnel and diluted with 15 mL of methylene chloride and 25 mL of water. The organic layer was extracted, dried with anhydrous sodium sulfate, and concentrated, under reduced pressure, furnishing 222 mg of 3a, 3b as a pale yellow oil (90% of theoretical). ¹H NMR (CDCl₃, 400 MHz, salient cis isomer, 3a) δ (ppm) 3.92 (m 2H), 2.98 (m, 2H), 2.81 (m, 2H), (m, 2H), 2.01 (m, 2H), 1.77 (m, 2H); ¹³C NMR (CDCl₃, 100 MHz salient cis isomer) δ (ppm) 114.23, 69.8, 30.2, 20.1.

Step 3: Synthesis of 2,2′-((2R,5S)-tetrahydrofuran-2,5-diyl)diacetic acid 4a and 2,2′-((2S,5S)-tetrahydrofuran-2,5-diyl)diacetic acid, 4b

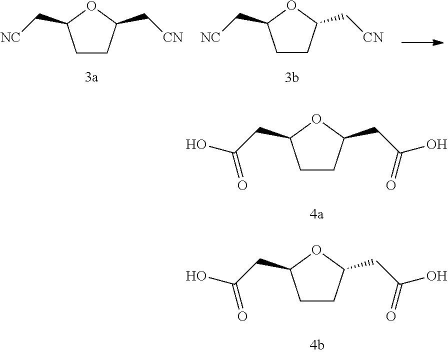 [SMILES: N#CC[C@@H]1CC[C@H](CC#N)O1 ; N#CC[C@@H]1CC[C@@H](CC#N)O1 ; O=C(O)C[C@H]1CC[C@@H](CC(=O)O)O1 ; O=C(O)C[C@@H]1CC[C@@H](CC(=O)O)O1]

Experimental: A 25 mL round bottomed flask equipped with an octagonal magnetic stir bar was charged with 200 mg of 3a, 3b (1.33 mmol), and 10 mL of 3N aqueous HCl. The mixture was stirred vigorously for 2 h, after which time an aliquot was removed and analyzed by ¹³C NMR (400 MHz, d⁶-DMSO). A salient signal at 173.4 ppm coupled with the absence of signature nitrile signal at 114.23 ppm was cogent proof that full conversion had occurred. Excess solvent was then removed in vacuo, affording 231 mg of 4a and 4b as beige solid (92%). ¹H NMR (D₂O, 400 MHz, salient cis isomer, 4a) δ (ppm) 4.01 (m 2H), 2.42 (m, 2H), 2.19 (m, 2H), (m, 2H), 1.90 (m, 2H), 1.61 (m, 2H); ¹³C NMR (D₂O, 100 MHz salient cis isomer) δ (ppm) 171.3, 76.5, 40.2, 30.6.

Example 2, demonstrates an iteration using an alternate sulfonate species, cyanide reagents, and/or solvents.

Step 1. Synthesis of ((2R,55)-tetrahydrofuran-2,5-diyl)bis(methylene) bis(4-methylbenzenesulfonate) and diastereomer

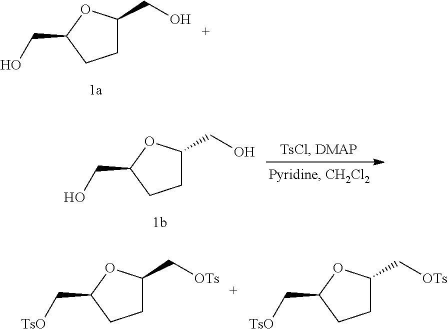 [SMILES: OC[C@@H]1CC[C@H](CO)O1 ; OC[C@@H]1CC[C@@H](CO)O1 ; Cc1ccc(S(=O)(=O)OC[C@H]2CC[C@@H](COS(=O)(=O)c3ccc(C)cc3)O2)cc1 ; Cc1ccc(S(=O)(=O)OC[C@@H]2CC[C@@H](COS(=O)(=O)c3ccc(C)cc3)O2)cc1]

Experimental: An oven dried, 25 mL single-neck round bottomed flask equipped with a ½″×⅛″ tapered PTFE coated magnetic stir bar was charged with 300 mg of THF-diols 1a and 1b (2.27 mmol), 866 mg of p-toluenesulfonyl chloride (tosyl chloride, 4.54 mmol), 550 μL of pyridine (˜3 eq.), 2.7 mg of dimethylaminopyridine (DMAP, 0.227 mmol) and 10 mL of anhydrous methylene chloride. The neck was capped with a rubber septum and a needle affixed to an argon inlet stirred vigorously overnight under an argon blanket. After this time, the solution was transferred to a 100 mL separatory funnel, diluted with 20 mL of methylene chloride, and washed three times with 10 mL of a 1N aqueous HCl solution. After each washing, the aqueous layer was removed, and residual organic phase dried with anhydrous magnesium sulfate then concentrated, after filtration, under reduced pressure, affording 922 mg of a light yellow solid, representing ((2R,5S)-tetrahydrofuran-2,5-diyl)bis(methylene) bis(4-methylbenzenesulfonate) and diastereomer (92% of theoretical). ¹H NMR (CDCl₃, 400 MHz, salient cis isomer) δ (ppm) 7.79 (d, J=8.0 Hz, 2H), 7.39 (d, J=8.0 Hz, 2H), 4.36 (m, 2H), 4.33 (m, 2H), 4.21 (m, 2H), 2.51 (s, 6H), 2.11 (m, 2H), 1.80 (m, 2H); ¹³C NMR (CDCl₃, 100 MHz salient cis isomer) δ (ppm) 146.7, 142.6, 132.1, 129.3, 84.0, 72.5, 30.6, 22.1

Step 2. Synthesis of 2,2′-((2R,55)-tetrahydrofuran-2,5-diyl)diacetonitrile and diastereomer

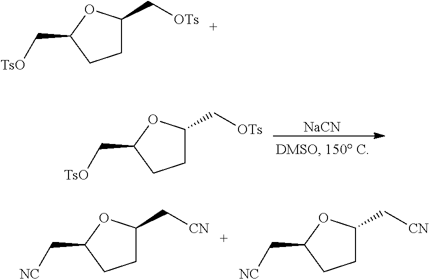

Experimental: An oven dried, 25 mL single-neck round bottomed flask equipped with a ½″×⅛″ tapered PTFE coated magnetic stir bar was charged with 700 mg of ((2R,55)-tetrahydrofuran-2,5-diyl)bis(methylene) bis(4-methylbenzenesulfonate) and diastereomer (1.59 mmol), and 5 mL of anhydrous DMF. The flask was capped with a rubber septum affixed to an argon and, while stirring and under argon, 438 μL of trimethylsilyl cyanide (3.50 mmol) was injected dropwise. The septum was then replaced with a reflux condenser attached to an argon inlet, and solution stirred vigorously at 150° C. overnight. After this time, an aliquot was removed and a portion spotted on a silica gel thin-layer chromatography plate abutting a spot from the THF diol starting material for comparison. The plate was developed using a 50% ethyl acetate in hexanes as the eluent, and after staining with cerium molybdate, the product mixture revealed one distinct spot, R_{fl}=0.58 (THF-diol ditriflate). No band was observed at R_{f}=0.41, corresponding to reactants, indicating that these had been entirely converted. The solution was transferred to a 50 mL separatory funnel and diluted with 15 mL of methylene chloride and 25 mL of water. The organic layer was extracted, dried with anhydrous sodium sulfate, and concentrated, under reduced pressure, furnishing 216 mg of a 2,2′4(2R,5S)-tetrahydrofuran-2,5-diyl)diacetonitrile and diastereomer as a pale yellow oil (91% of theoretical). ¹H NMR and ¹³C NMR spectra were consistent with those described previously.

Step 3. Synthesis of 2,2′4(2R,5S)-tetrahydrofuran-2,5-diyl)diacetic acid and diastereomer

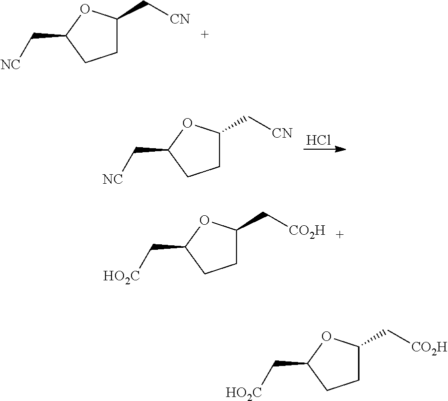

The same reaction provisions to prepare these compounds were as previously detailed.

B. Synthesis of THF-2,5-dicarbaldehydes by Partial Reduction of THF-2,5-dinitriles

The process for making dicarbaldehydes is similar to that described for the diacids, until the third reaction step. Instead of oxidization, the THF-2,5-dinitrile is partially reduced. The following examples demonstrate some different approaches to convert the diacetonitrile to a corresponding aldehyde.

### Example 1

Conversion of 2,2′-((2R,5S)-tetrahydrofuran-2,5-diyl)diacetonitrile and diastereomer to 2,2′4(2R,5S)-tetrahydrofuran-2,5-diyl)diacetaldehyde and diastereomer using diisobutylaluminum hydride at low temperature.

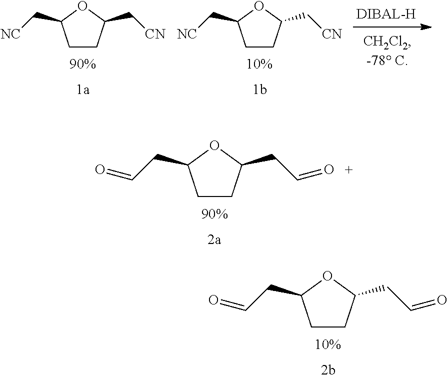

Experimental: A flame-dried, 25 mL round bottomed flask equipped with a magnetic stir bar was charged with 200 mg of a 9:1 mixture of 2,2′-((2R,55)-tetrahydrofuran-2,5-diyl)diacetonitrile 1a and 2,2′-((2S,5S)-tetrahydrofuran-2,5-diyl)diacetonitrile 1b (1.33 mmol) and 10 mL of anhydrous dichloromethane. The flask was capped with a rubber septum affixed to an argon inlet, then immersed in a saturated dry ice/acetone slurry (−78° C.). While stirring and under argon, 1.33 mL of a 1 M diisobutylaluminum hydride solution in methylene chloride was added dropwise over 5 min and the reaction continued at −78° C. for 1 hour. After this time, the dry ice/acetone bath was removed, and reaction was quenched with a minimum amount of water. Solids were filtered, and the permeate concentrated in vacuo over 72 hours, producing a 198 mg of a clear, colorless oil (95% of theoretical). ¹H NMR (400 MHz, CDCl₃, salient cis product) δ (ppm) 9.75 (m, 2H), 4.02 (m, 2H), 2.71 (m, 2H), 2.33 (m, 2H), 1.84 (m, 2H), 1.57 (m, 2H); ¹³C NMR (100 MHz, CDCl₃, salient cis product) δ (ppm) 198.4, 73.2, 50.7, 29.7.

### Example 2

Synthesis of 2,2′-((2R,5S)-tetrahydrofuran-2,5-diyl)diacetaldehyde and diastereomer from 2,2′-((2R,5S)-tetrahydrofuran-2,5-diyl)diacetonitrile and diastereomer using Raney Nickel in formic acid.

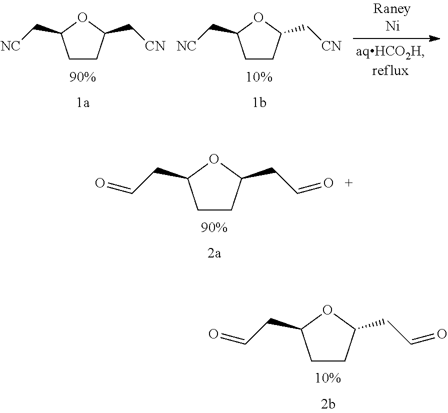

Experimental: A 100 cc round bottomed flask equipped with a magnetic stir bar was charged with 1 g of a 9:1 mixture of 2,2′-((2R,55)-tetrahydrofuran-2,5-diyl)diacetonitrile 1a and 2,2′4(25,5S)-tetrahydrofuran-2,5-diyl)diacetonitrile 1b (6.66 mmol), 2 g of Raney nickel, and 50 mL of ˜88% formic acid. The flask was then outfitted with an Allihn condenser and the mixture heated to reflux for 2 h. After this time, the solution was cooled to ambient temperature, then vacuum filtered using a Buchner funnel The remnants were then concentrated under reduced pressure over 48 hours, resulting in a loose, colorless oil, that was then suspended in water and heated to a boil for 30 min. After this time, excess water was removed and the crude product charged to a pre-fabricated silica gel column, which effectively sequestered 586 mg of the title compounds 2a and 2b (56% of theoretical) as clear, colorless oils using a hexanes/ethyl acetate gradient mobile phase (50% to 100% ethyl acetate). ¹H and ¹³C NMR were congruent with the aforementioned product analysis delineated in Example 1, above, of the aldehyde conversion.

### Example 3

Partial catalytic reduction of 2,2′4(2R,5S)-tetrahydrofuran-2,5-diyl)diacetonitrile and diastereomer to 2,2′-((2R,5S)-tetrahydrofuran-2,5-diyl)diacetaldehyde and diastereomer under mild, hydrolytic conditions.

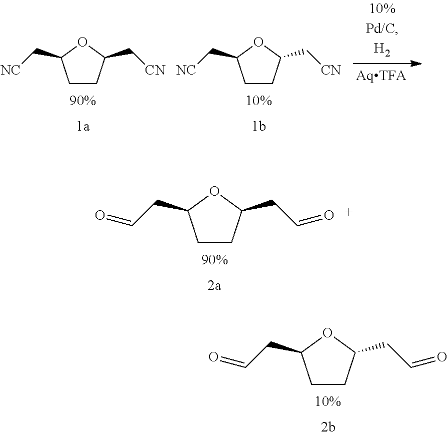

Experimental: A 250 cc Hasteloy autoclave with an overhead stirrer was charged with 2 g of a 9:1 mixture of 2,2′-((2R,5S)-tetrahydrofuran-2,5-diyl)diacetonitrile 1a and 2,2′-((25,55)-tetrahydrofuran-2,5-diyl)diacetonitrile 1b (13.32 mmol), 1 g of 10% Pd/C, and 50 mL of a 50% aqueous trifluoroacetic acid (TFA) solution. The vessel was sealed, purged ×3 with volumes of H₂ corresponding to 250 psi then pressurized with H₂ until the gauge read 200 psi. The mixture was stirred at 40° C. overnight at room temperature, after which time the excess catalyst removed by vacuum filtration, and residual solution evaporated under reduced temperature, affording a colorless, loose crude oil. This was diluted with a minimal amount of methylene chloride and charged to a prefabricated silica gel column which effectively sequestered 1.02 g of the title compounds 2a and 2b (49% of theoretical) as clear, colorless oils using a hexanes/ethyl acetate gradient mobile phase (50% to 100% ethyl acetate). ¹H and ¹³C NMR were congruent with the aforementioned product analysis delineated in Example 1.

C. Synthesis of THF-2,5-diamines by Complete Reduction of THF-2,5-diacetonitriles

As with the foregoing examples relating to oxygenated products, THF-diol is first subjected to sulfonation and derivatized. The resulting THF-sulfonate is then reacted with a nucleophile, such as cynide, to generate a diacetonitrile species. The following presents embodiments of the diamine synthesis process, which has two viable pathways to complete reduction of the diacetonitrile species.

Ex. Route 1: Synthesis of 2,2′-((2R,5S)-tetrahydrofuran-2,5-diyl)diethanamine 2a and 2,2′-((2S,5S)-tetrahydrofuran-2,5-diyl)diethanamine 2b from bHMTHFs 1a and 1b using a metal hydride.

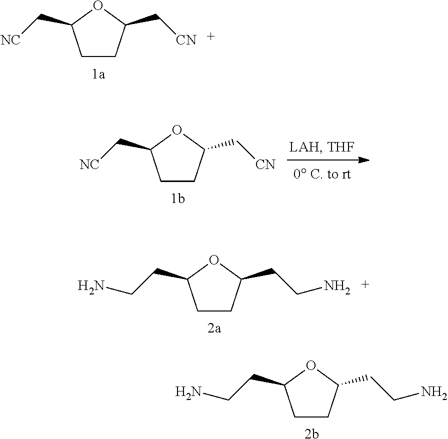

Experimental: A flame-dried, single necked 10 mL round bottomed flask equipped with a PTFE coated magnetic stir bar was charged with 125 mg of a 9:1 mixture of 1a and 1b (0.832 mmol) and 5 mL of anhydrous THF. The homogeneous mixture was cooled to −10° C. in a saturated brine/ice bath, and, while stirring, 1.67 mL of a 1M solution of lithium aluminum hydride in THF (LAH) was added dropwise over 10 min (1.67 mmol). After complete addition, the ice bath was removed and reaction continued at room temperature for 2 h. After this time, solids were filtered, and the filtrate washed with 2 mL of a 0.5M HCl solution. Excess solvent was then removed in vacuo, affording 121 mg of 2a and 2b as a light yellow syrup (92% of theoretical). ¹H NMR (400 MHz, CDCl₃, salient cis signals) δ (ppm) 4.95 (s, 4H), 3.51 (m, 2H), 2.79 (m, 4H), 2.03 (m, 2H), 1.70-1.67 (m, 6H); ¹³C NMR (100 MHz, CDCl₃, salient cis signals) δ (ppm) 80.8, 40.5, 37.9, 32.0 ppm.

Ex. Route 2: Synthesis of 2,2′-((2R,55)-tetrahydrofuran-2,5-diyl)diethanamine 2a and 2,2′-((2S,5S)-tetrahydrofuran-2,5-diyl)diethanamine 2b from bHMTHFs 1a and 1b via catalytic hydrogenation.

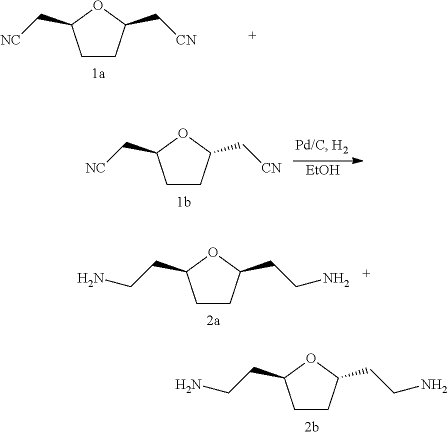

Experimental: A 300 cc stainless steel Parr reactor vessel was charged with 250 mg of a 9:1 mixture of 1a and 1b (1.65 mmol), 200 mg of 10% Pd/C and 100 mL of absolute ethanol. The vessel was affixed to the reactor apparatus, sealed, purged ×3 with volumes equal to 1000 psi of hydrogen gas (H₂), and pressurized to 1200 psi with H₂. While overhead stirring at 500 rpm, the hydrogenation reaction proceeded for 2 h at room temperature. After this time, solids were filtered, and surplus solvent removed under reduced pressure, affording 258 mg of 2a and 2b as a clear, viscous oil (98% of theoretical). ¹H NMR (400 MHz, CDCl₃, salient cis signals) δ (ppm) 4.95 (s, 4H), 3.51 (m, 2H), 2.79 (m, 4H), 2.03 (m, 2H), 1.70-1.67 (m, 6H); ¹³C NMR (100 MHz, CDCl₃, salient cis signals) δ (ppm) 80.8, 40.5, 37.9, 32.0 ppm.

The present invention has been described in general and in detail by way of examples. Persons of skill in the art understand that the invention is not limited necessarily to the embodiments specifically disclosed, but that modifications and variations may be made without departing from the scope of the invention as defined by the following claims or their equivalents, including other equivalent components presently known, or to be developed, which may be used within the scope of the present invention. Therefore, unless changes otherwise depart from the scope of the invention, the changes should be construed as being included herein.

1-26. (canceled)

27. A process for preparing either an oxygenated or amine product from 2,5-bis(hydroxymethyl)-tetrahydrofuran (bHMTHF), comprising: a) derivatizing bHMTHF using a sulfonate to generate a tetrahydrofuran-2,5-diyl-bis(methylene)-bis(sulfonate); b) displacing at least a sulfonate leaving group from said tetrahydrofuran-2,5-diyl-bis(methylene)-bis(sulfonate) with a nucleophile; and either c) hydrolyzing fully with a strong acid having a pKa of ≦0 to generate a di-acid, or d) reducing partially to generate a di-aldehyde, or e) reducing fully to generate a diethyl-amine.

28. The process according to claim 27, wherein said di-acid is a tetrahydrofuran-2,5-diyl-diacetic acid, and said di-aldehyde is a tetrahydrofuran-2,5-diacet-aldehyde.

29. The process according to claim 27, wherein said sulfonate is at least one of the following: methanesulfonate, trifluoromethanesulfonate, ethanesulfonate, benzenesulfonate, and p-toluenesulfonate.

30. The process according to claim 27, wherein said derivatization with said sulfonate involves a nucleophilic base catalyst that is a nitrogen-centered compound.

31. The process according to claim 27, wherein said displacing reaction of said sulfonate leaving group involves a cyanide nucleophile.

32. The process according to claim 27, wherein said displacing reaction of said sulfonate leaving group from said tetrahydrofuran-2,5-diyl-bis(methylene)-bis(sulfonate) generates a diacetonitrile derivative.

33. The process according to claim 32, wherein said diacteonitrile derivative is a cis or trans isomer:

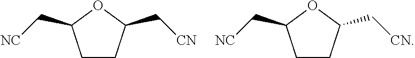 [SMILES: N#CC[C@@H]1CC[C@H](CC#N)O1 ; N#CC[C@@H]1CC[C@@H](CC#N)O1]

34. The process according to claim 27, wherein said derivatization of said bHMTHF is performed in a solution of solvent having a boiling point ≧110° C., and a relative permittivity ≧εᵣ 25.

35. The process according to claim 34, wherein said solvent has a boiling point ≧120° C., and a relative permittivity ≧εᵣ 30.

36. The process according to claim 27, wherein said displacing reaction of said sulfonate leaving group is performed in an aprotic solvent.

37. The process according to claim 36, wherein said aprotic solvent is at least one of the following: dimethylsulfoxide (DMSO), N,N-dimethylformamide (DMF), N,N-dimethylacetamide (DMA), N-methyl-2-pyrrolidone (NMP), hexamethylphosphoramide, and nitrobenzene.

38. The process according to claim 27, wherein said strong acid is at least one of the following: aqueous hydrochloric, hydrobromic, hydroiodic, perchloric, sulfuric, p-toluenesulfonic, triflic, methanesulfonic, and benzenesulfonic acids.

39. The process according to claim 27, wherein said process is conducted in a temperature range from about 10° C. to about 180° C. for derivatizing with a sulfonate species other than trifluoromethanesulfonate.

40. The process according to claim 27, wherein said process is conducted in a temperature range from about −25° C. to about 0° C. for derivatizing with trifluoromethanesulfonate.

41. The process according to claim 27, wherein said process is conducted in a temperature range from about 0° C. to about 200° C. for displacing with said nucleophile.

42. The process according to claim 41, wherein said process is conducted in temperature range from about 125° C. to about 180° C. for displacing with said nucleophile.

43. The process according to claim 27, wherein a 9:1 diastereomeric mixture of cis and trans bHMTHFs derived from HMF is converted to a) a 9:1 diastereomeric mixture of THF-2,5-diacetic acid; or b) a 9:1 diastereomeric mixture of THF-2,5-diacetaldehyde; or c) a 9:1 diastereomeric mixture of THF-2,5-diamine.

44. The process according to claim 27, wherein said THF-2,5-diacetonitrile either is reduced partially to said THF-2,5-diacetaldehyde using at least: 1) a hindered organo-metalic hydride or 2) a catalyst, or is reduced fully to said THF-2,5-diamine using at least: 1) a hindered organo-metalic hydride or 2) a catalyst.

45. The process according to claim 44, wherein said catalyst is: a) a nickel catalyst in absence of hydrogen or b) a palladium catalyst.

46. An oxygenated or amine product prepared from 2,5-bis(hydroxymethyl)-tetrahydrofuran (bHMTHF) comprising at least one of the following: a cis or trans isomer of tetrahydrofuran-2,5-diacetic acid, as: 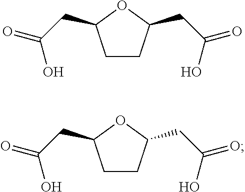 [SMILES: O=C(O)C[C@H]1CC[C@@H](CC(=O)O)O1 ; O=C(O)C[C@@H]1CC[C@@H](CC(=O)O)O1]
a cis or trans isomer of tetrahydrofuran-2,5-diacetaldehyde, as: 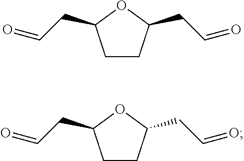 [SMILES: O=CC[C@@H]1CC[C@H](CC=O)O1 ; O=CC[C@@H]1CC[C@@H](CC=O)O1]
or a cis or trans isomer of tetrahydrofuran-2,5-diamine, as: [chemical structure]
